(19) United States
(12) Patent Application Publication
(10) Pub. No.: US 2002/0116750 A1
(43) Pub. Date: Aug. 29, 2002

(54) Visual discomfort relief

(76) Inventor: Donald R. Korb, Boston, MA (US)

Correspondence Address: DIKE, BRONSTEIN, ROBERTS AND CUSHMAN,, INTELLECTUAL PROPERTY PRACTICE GROUP, EDWARDS & ANGELL, LLP., P.O. BOX 9169, BOSTON, MA, 02209, US

(21) Appl. No.: 09/920,290

(22) Filed: Jul. 31, 2001

Related U.S. Application Data
(60) Provisional application No. 60/222,090, filed on Aug. 1, 2000.

(51) Int. Cl. A61F 9/02

(52) U.S. Cl. 2/428

(57) ABSTRACT

The invention relates to a method for avoiding the onset of eye discomfort while performing prolonged near point activities such as use of a computer or reading. The method of the invention comprises creation of a high periocular relative humidity over the surface of the eyes when engaged in prolonged near point activities. Preferably, the relative humidity is maintained at least at eighty percent and preferably at least at ninety percent. The increased humidity maintains the thickness of the lipid layer during such activity thereby reducing evaporation from the tear film. The high periocular relative humidity is desirably achieved using frames over the eye to encase the eye in a confined but controlled atmosphere.

This application claims the benefit of U.S. Ser. No. 60/222,090, filed Aug. 1, 2000.

### BACKGROUND OF THE INVENTION 1. Field of the Invention

This invention relates to the relief of eye discomfort resulting from prolonged close visual activity such as computer use, reading or close inspection. More specifically, this invention provides a means for maintaining the thickness of the lipid layer over the corneal surface when performing prolonged close visual tasks thereby eliminating or reducing the cause of discomfort associated with such activity.

2. Description of the Prior Art

It is known in the art that eye discomfort and related symptoms occur when working with visual display terminals [VDTs]. To a lesser extent, similar symptoms occur when reading for a prolonged period of time or engaging in other close concentrated activity such as assembly line work, especially assembly of small parts and inspection. Symptomatology of this nature is reported in a 1982 study which found that eye discomfort was the most common symptom experienced with visual display terminal [VDT] use; Grundy J W, Rosenthal S G. VDTs on site. Ophthal Opt 1982;22:500-12. In 1991, a study by the National Institute of Occupational Safety & Health reported that 88% of all individuals working with a VDT screen for two or more hours per day experienced visual or muscular complaints and emotional difficulties; National Institute for Occupational Safety Health, NIOSH publication on VDTs, Cincinnati, Ohio: U.S. Department of Health and Human Services, Public Health Service, Center for Disease Control, National Institute for Occupational Safety and Health, 1991. This study estimated that up to 40 million workers suffer symptoms of visual stress when using a VDT for three or more hours daily. The reported symptoms included blurred vision, burning or tired eyes, and loss of focus and headaches. A 1991 Harris poll identified computer eyestrain as the number one job-related complaint in the U.S. work force; Harris Poll of 1991 quoted in: Branscum D: Clear-eyed computing. MAC World 1992;9:69-71. A mail survey of 5,686 randomly selected practicing optometrists (25.3% response rate) found that on the average, 14.25% of their patients obtained an eye examination because of symptoms primarily related to computer use; Sheedy J E: VDT's and vision complaints: A survey. Information Display 1992;8:20-23. The four primary symptoms reported, in their order of frequency, were eyestrain, headaches, blurred vision, and dry or irritated eyes. This survey suggests that approximately 10 million of the 70 million general eye examinations given annually in the United States are due to eye symptoms related to computer use. These studies reinforce the daily experiences of optometrists and ophthalmologists involved with general eye examinations of patients suffering computer-related problems.

The problems described above are exacerbated by prolonged observation of a VDT screen. The problem is also encountered when reading or when engaged in other close visual activities. The severity of discomfort and the time required for the onset of discomfort varies from individual to individual and is dependent upon the activity performed. However, in general, the time is shorter when working with a VDT compared to other activities such as reading.

Eye care professionals, the computer industry, and other parties have dedicated significant effort to treatment of the symptoms and problems associated with computer use. Initial efforts were directed to improving glare reduction and display image quality. These efforts have at best provided only partial relief of the symptoms associated with these activities. A study by Salibello & Nilsen in 1995 reported that there were no statistically significant differences in proportions of symptomatic and non-symptomatic individuals based on the VDT design when comparing old vs. recent designs, Salibelo C, Nilsen E: is there a typical VDT patient? A demographic analysis. J. Am Optom Assn 1995; 66; 479-83. Thus, the cause of the discomfort is not related to VDT design.

Common sense approaches to minimizing the effects of lengthy VDT use were suggested in 1995. The approaches included computer positioning, adjustable workstations, adjustable VDT brightness and contrast, and glare reduction filters. While generally helpful, these approaches have not significantly alleviated the discomfort for the majority of sufferers, Sheedy J E: VDT-related eye problems cost U.S. $2 billion annually. AOA News 1995.

Specialized eye examinations have been developed to specifically provide the optimal visual correction at the distance and vertical position of the VDT screen from the eye. However, despite these sophisticated examinations, eye discomfort remains a widespread and chronic problem for a large percentage of computer users.

Additional treatments for visual discomfort associated with prolonged computer use are numerous, but there has been no significant change in the problem over the past ten years. Recommendations include special ophthalmic lenses and eyeglasses, blue light filters, screens to attach to the VDT to bleach radiation, mechanical accupressure products, vision therapy and additional room lights. An eye drop medication to help wet the eyes during computer use has been attempted, but this eye drop has not provided significant relief for the majority of those suffering eye discomfort. A web site, www.drsheedy.com/healtheffects.htm1/, addressing the problem is available with extensive information presented. The magnitude of information concerning the problem on this web site and other similar sites confirm the severity of the problem. Consequently, despite all efforts to date, visual discomfort remains one of the primary public health problems of office workers commanding not only the attention of the eye care profession, but also that of public health and governmental agencies. The societal costs are enormous if one considers absenteeism, reduced work efficiency, work “burnout,” professional consultation as a result of symptoms, and general morale in the workplace.

The magnitude of the problem described above has resulted in government intervention. The most intensive government intervention has been in Europe where the European Council has passed a directive (EC Directive 90/270/EEC), which states that all VDT workers are entitled to an “eye and eyesight test” before commencing work, at regular intervals thereafter, and whenever they have visual difficulties. The EC Directive further states that if the results of the examination “show that it is necessary and if a normal corrective appliance cannot be used, workers must be provided with special corrective appliance appropriate for the work concerned.” However, despite the EC actions and widespread use of corrective eyeglasses, the problem remains widespread and chronic in Europe for a large percentage of computer users. While there have been more than 200 legislative efforts regarding VDT worker health and safety in the U.S., none have been successfully passed.

### Definitions

The following definitions have been adopted for use in the discussion that follows:

The term “near point activity” means visual activity within a working distance of between 12 and 36 inches between the individual's eyes and object read or observed. For example, near point activity could include computer use, reading, assembly and close inspection.

The term “NPIEDS” is used as an acronym for near point induced eye discomfort syndrome resulting from prolonged near point activity. It can take the form of any one or more of visual, eye, and related neurological symptoms as described above.

The term “VDT” is used herein to mean video display terminal.

### SUMMARY OF THE INVENTION

The subject invention is based upon a combination of observations and discoveries. One observation is that there is a significant decrease in the thickness of the lipid layer over the surface of the eye when an individual is engaged in prolonged near point activity, probably caused by a decrease in blinking during periods of intense concentration. The tear film is comprised of three layers, the outer lipid layer, the middle aqueous layer and the inner mucus layer. The thin lipid layer (average thickness 60-120 nm) makes contact with the ambient atmosphere and is anterior to the aqueous layer. The purpose of the lipid layer is to prevent evaporation of the aqueous layer from the eye. The aqueous layer is a relatively thick layer (4-8 μ) that functions as a lubricating, cushioning and protective layer for the epithelial surface of the cornea and provides a renewable optical quality surface required for optimal vision. The innermost layer of the tear film is the mucus layer, which allows the aqueous layer to form over the epithelial surface of the cornea.

Without the lipid layer, the rate of evaporation of fluid from the aqueous layer to the atmosphere is increased by a factor of fifteen times; Mishima S, Maurice D M. The oily layer of the tear film and evaporation from the corneal surface. Exp Eye Res 1961:1:39-45. This results in a significant thinning of the aqueous layer with a concomitant increase in discomfort. Consequently, where it has been observed that thinning of the lipid layer occurs when engaged in near point activity, it is believed that this results in evaporation of the aqueous layer resulting in NPIEDS.

A second factor leading to the invention is the recognition that thinning of the lipid layer may be substantially reduced during prolonged near point activity if a high humidity atmosphere is maintained over the surface of the eyes when engaged in this activity. In this respect, it has been found that the time for the onset of NPIEDS associated with near point activity can be substantially prolonged when the relative humidity over the surface of the eye, periocular relative humidity, is at least 80%, preferably in excess of 90% and more preferably, close to or at 100%.

A third aspect of the invention is provision for a means to maintain a high periocular humidity over the eye when engaged in prolonged near point activity. In a preferred embodiment of the invention, the means to maintain high humidity comprises glasses or goggles having a soft rubber like flange around their rim designed to create an almost closed environment over the eye while permitting the transfer of oxygen and exchange of gases to the eye so as to avoid oxygen deficiency and physiological compromise of the eye without fogging of the goggle's lenses.

### DESCRIPTION OF THE PREFERRED EMBODIMENTS

As aforesaid, the invention comprises provision of means for maintaining a high periocular relative humidity over the surface of the eyes during prolonged near point activity to extend the period during which one can engage in such activity without experiencing NPIEDS.

Prior to making the discovery that high periocular relative humidity prolongs the onset of NPIEDS, the inventor performed research on the causes of NPIEDS. Relying upon conventional teaching, investigations were performed relating to numerous specialized optical combinations of lenses and custom prisms designed for the individual's particular working distance and the positioning of the head and computer. These approaches were only productive if there was an obvious pre-existing visual deficiency at the near point activity distance of 12 to 36 inches. However, less than 15% of those suffering from NPIEDS were found to have a visual condition of a magnitude at this distance, which could account for NPIEDS. The study indicated that of those suffering from NPIEDS, 85% did not have an obvious visual deficiency at their specific working distance of 12 to 36 inches. Moreover, the discomfort for these individuals could not be alleviated by any lens prescription, even by customizing the prescription to minimize the visual effort at the individuals' specific working distance and positioning. Therefore, it was concluded that ophthalmic prescriptions were not a universal answer for the large majority of those suffering NPIEDS.

The inventor then performed research on the effect of near point activity on blinking to determine if this might impact NPIEDS. It is known that the blink rate is not a constant for any individual and that there is a large difference in blink rate between individuals; York M, Ong J, Robbins J C. Variation in blink rate associated with contact lens wear and task difficulty. AM J Optom Arch Am Acad Optom 1971;48:461-6; Stem J A, Walrath L C, Goldstein R. The endogenous eye-blink. Psychophysiology 1984;21 :22-23. The usually accepted range of blink rate is 8 to 15 blinks per minute under normal circumstances with an average of 12 blinks per minute; Carney L G, Hill R M. The nature of normal blinking patterns. Acta Ophthalmol (Kbh) 1982;60:427-33. Blink rate is known to decrease during concentrated near point activity. For example, one study has shown that the blink rate decreased from 15 blinks per minute to 4 blinks per minute when the visual task was changed from watching a film to underlining letters on the page of a text; York M, Ong J, Robbins J C. Variation in blink rate associated with contact lens wear and task difficulty. Am J Optom Arch Am Acad Optom 1971;48:461-6. Other studies have shown the blink frequency decreases during VDT use by a factor of three to five times; Yaginuma Y, Yamada H, Nagai H: Study of the relationship between lacrimation and blink in VDT work. Ergonomics 1990;33:799-809; Patel S, Henderson R, Bradley L, Galloway B, Hunter L. Effect of visual display unit use on blink rate and tear stability. Optom Vis Sci 1991;68:888-92; and Tsubato K, Hakomori K. Dry eyes and video display terminals. N Engl J Med 1993;328:584.

It is accepted that blinking is necessary to maintain the wetting of the ocular surfaces of the front of the eye and to prevent dry eye symptoms. The aforementioned 1992 survey revealed that the most frequent symptom related to computer use was eyestrain, followed by headaches, blurred vision, and then dry or irritated eyes. Blinking exercises have been suggested for these symptoms; Collins M, Heron H, Larsen R, Lindner R., Blinking patterns in soft lens wearers can be altered with training. Am J Optom Physiol Opt 1987;64:100-3; and Lowenstein E. VDT users: Improve comfort with the blink of an eye. Polymer Technology Corporation, Cambridge, Mass., 1988. Thus, it is acknowledged that the act of blinking is necessary to maintain appropriate wetting of ocular surfaces and it has been established that a three to five fold decrease in blinking is associated with computer use. However, there is no validated information as to what role decreased blinking plays in NPIEDS and whether increasing the frequency of blinking alleviates the symptoms. Nevertheless, it would appear to follow that increasing a depressed blink rate should help to both wet the front of eye and to alleviate the symptoms of NPIEDS.

To evaluate the effect of increasing blink frequency during computer activities on NPIEDS, the inventor hereof conducted several studies. The first study used a metronome to provide an audible signal at which time the subject was instructed to blink. The audible signal was set for 10 seconds. Subjects were instructed to blink at the audible signal, but despite reported compliance, there was no statistically significant improvement in the magnitude of eye discomfort. The second study utilized visual cues to cause the individual to blink. The cues were displayed by computer programming whereby at 10 to 20 seconds' intervals, a message was flashed requesting the individual to blink. Despite reported compliance, there was no statistically significant improvement in the eye discomfort. From these studies, it was concluded that though computer use depresses blink rate and appears to be a factor in creating symptoms similar to those suffered by patients with dry eyes, attempting to improve blinking by providing conscious stimuli does not appear to provide significant relief for most computer users.

In addition to the inventor's studies, independent studies have attempted to investigate whether there are changes in objective ocular findings that occur as a result of computer use when NPIEDS is present. However, no change in any objective finding such as tear film stability, tear film chemistry, and/or desiccation of the corneal tissue has been found. In fact, Patel et al.; Patel S., Henderson, R., Bradley L., Galloway, B., Hunter L., Effect of visual display unit use on blink rate and tear stability. Optom Vis Sci 1991; 68;888-92, reported that a reduced blink rate during computer use, did not alter the stability of the precorneal tear film, which remained normal despite a reduction in the blink rate of approximately 5 times. Ousler et al, in a poster presented at the Association for Research in Vision and Ophthalmology at the 1999 Annual Meeting in Fort Lauderdale, Fla., on May 9 to 14, 1999 reported use of a controlled “adverse” environment”, now know as a “Dry Eye Room” with low humidity of 7%. This is an unrealistic environment for working on a computer-even in the winter it is usually at least 20% indoors. Only minor changes were found for break-up time and an increase in keratitis. A staining increase was large and the blink rate was depressed by 57%. However, the environment was not the same as Patel's, nor was the test realistic in terms of the environment used.

The observation that there were no detectable ocular findings of an adverse nature for subjects severely suffering from NPIEDS appeared counter-intuitive to the inventor. Therefore, the inventor investigated an area not previously receiving attention, change in the thickness of the lipid layer while engaged in prolonged near point activity. As is known, the lipid layer is responsible for preventing evaporation of fluid from the aqueous layer over the eye.

The data for the study of lipid layer thickness during computer use is presented in the following Table I.

TABLE I
DECREASE IN LIPID LAYER THICKNESS
WITH LONGITUDINAL COMPUTER USE

| | Baseline Lipid Layer | | | |
|---|---|---|---|---|
| | Thickness Prior to Computer | Lipid Layer Thickness | | |
| | Use | Following Computer Use | | |
| Subject | [in nanometers] | 1 HR | 2 HRS | 3 HRS |
| TD | 150 | 115 | 90 | 75 |
| AF | 120 | 90 | 75 | 75 |
| AW | 90 | 60 | ≦45 | ≦45 |
| SH | 90 | 60 | ≦60 | ≦60 |
| KL | 75 | ≦45 | ≦45 | ≦45 |

These above data demonstrate that lipid layer thickness decreases with prolonged longitudinal computer use, frequently to 60 nm or less. It is known in the art that a lipid layer thickness of 60 nm or less correlates with dry eye symptoms. It is also known that lipid layer thickness can be increased if the periocular relative humidity is raised to high levels. Both are reported by Korb D R, Greiner J V, Esbah R, Finnemore V M, Whalen A C, The effect of periocular humidity on the tear film lipid layer. Cornea 1996; 15:129-34. The discovery of lipid layer thinning while engaged in prolonged near point activity and the knowledge that a relatively high humidity maintains lipid layer thickness motivated the inventor to study the effect of high levels of periocular relative humidity when engaged in prolonged near point activity.

The study referred to above involved creation of computer induced eye discomfort by use of a high concentration task on a computer VDT. After eye discomfort is induced, a device was fitted over the eyes to create a high level of periocular relative humidity to determine if the high humidity would alleviate the discomfort. Several different eye-covering devices were used. The first utilized standard anti-fog swim goggles that created individual watertight/airtight chambers in front of each eye. The swim goggles used were manufactured by Speedo. A second device was a swim mask that created a single watertight chamber in front of both eyes. The swim mask used was an anti-fog treated Aqua Sphere Seal mask distributed by U.S. Divers/Aqua Lung. A third device used was a commercially produced frame for outdoor use designed to fit close to the face and minimize pollutants and wind from reaching the ocular surfaces. This frame was labeled Lazor and distributed by Zeal Optics and had a small margin around the rim that permitted release of water vapor to the atmosphere.

To conduct the study, it was necessary to develop a standardized computer task to eliminate variables and maintain the subject's attention. A task found to be of interest to all subjects and able to maintain the subject's attention was a computer game called “Taipei”. This is a game in which tiles are matched until all matching has been completed and there are no free tiles. Certain computer activities, such as word processing, do not require steady state high levels of concentration. However, other computer activities require a significantly higher level of concentration such as data retrieval, research and precision reading. The Taipei game was found to simulate these concentrated activities.

The administration of a test to investigate eye comfort during computer use requires establishing an eye comfort rating scale that accurately portrays the subjective experience of the computer user. The following letter rating scale was adopted to grade eye comfort:

| Rating | Status | Description |
|---|---|---|
| A | Optimal | Eyes totally comfortable and without sensation. No eye discomfort and |
| | | no eye fatigue even under prolonged computer or reading activities. |
| | | Ability to work on computer for four or more hours without rest. |
| B | Comfortable | Eyes comfortable and would not notice eyes unless one thought of them - |
| | | then would have slight sensation prior to or upon blinking. No |
| | | compromise to computer or reading activities, and able to work on |
| | | computer for four or more hours without rest. |
| C | Awareness | Mild eye discomfort occurs after 1-3 hours, which is then, present the |
| | | majority of the time. Results in desire to alleviate by eye drops, blinking |
| | | or eye closure, but only slight intermittent compromise to computer or |
| | | reading activities. |
| D | Discomfort | Eye discomfort is at level to compromise computer or reading activities. |
| | | Unable to maintain prolonged computer or reading activities for longer |
| | | than 30 minutes without closing eyes, rest periods or other treatment. |
| | | One or more of the following subjective eye symptoms are present: |
| | | burning, aching, tearing, and blinking results in a scratchy and/or gritty |
| | | feeling. |
| F | Severe | Eye discomfort is at level to severely compromise computer or reading |
| | discomfort | activities. Unable to function for longer than 15 minutes without closing |
| | | eyes, rest periods or other treatment. One or more of the following |
| | | subjective eye symptoms are present: burning, aching, and tearing, |
| | | constant irritation, and blinking results in scratchy and/or gritty feeling. |

The specific steps used to conduct the study follow:

1. All subjects were required to be computer literate and to use a computer at least 1 hour daily. Subjects were not selected for study if an eyeglass correction was required for computer use. All subjects were then asked to evaluate their usual eye comfort during routine computer use utilizing the five-grade scale, from A to F. The study was conducted in a normal work environment, in a room 12 feet by 12 feet, with relative humidity between 35 and 40%.

2. Prior to the subject being seated at the computer, the subject was presented with a grading scale and asked to grade their eye comfort prior to computer use.

3. The subject was then seated at the computer and instructed in the use of the game Taipei. They were advised that they would be asked to grade their eye comfort.

4. The usual range of times for one game is 5 to 10 minutes. The subject was instructed to immediately continue with consecutive games until the examiner determined that the purpose of the study was completed. The subject was not advised that eye comfort might change during the study.

5. The desired end point was for the eye comfort rating to decline at least 1 full grade (e.g., B to C), and preferably two grades, so that a significant decrease in eye comfort could be established.

6. The examiner started the subject playing the game, continued to observe, and at 10 minutes intervals asked the subject to rate their eye comfort, and then recorded the data. The game was continued until eye comfort had decreased two or more grades, however this phase was terminated after 40 minutes regardless of the subject's performance. This was deemed necessary because of the possibility of excess fatigue, which could influence the patient in the second phase of the study.

7. Upon completing the requirements of phase 1, a swim goggle, as previously described, was immediately adjusted to the face without a rest period. The visual portion of the goggle was treated with an anti-fog preparation.

Immediately upon the fitting of the goggle, the comfort rating was again recorded. (A time interval of 30 to 120 seconds was required for the placement of the goggle and the re-initiation of the playing of the game.)

8. The subject then returned to precisely the same game activity and the prior steps were repeated for 40 minutes or until the eye comfort had returned to the baseline level prior to playing Taipei.

9. The same procedure was utilized for the swim mask and the Lazor wraparound eyeglasses. Only one study was performed on any one day.

The data for these studies of the effects of high humidity on NPIEDS are presented in Table II.

TABLE II
EYE COMFORT RATINGS PRIOR TO AND WHEN PLAYING
COMPUTER GAME WITH AND WITHOUT GOGGLES

| | | | | | | | Ratings Playing Taipei While Wearing | | | | |
|---|---|---|---|---|---|---|---|---|---|---|---|
| | | | | | | | Swim Goggles Creating High Periocular | | | | |
| | Usual | Baseline | Ratings Playing Taipei in | | | | Relative Humidity | | | | |
| | Ratings | Ratings | Ambient Room 35-40% | | | | Immmediately | | | | |
| | During | Prior to | Relative Humidity | | | | Upon | | | | |
| | Routine | Playing | 10 | 20 | 30 | 40 | Wearing | 10 | 20 | 30 | 40 |
| Subject | Use | Taipei | min | min | min | min | Goggles | min | min | min | min |
| TD | B⁻ | A | B | C | C⁻ | C⁻ | C⁻ | B | A | A | NC |
| EB | D | B | B⁻ | C⁺ | C | C⁻ | C⁻ | C | C⁺ | B | NC |
| VMF | C | B | B | C | C | C⁻ | C⁻ | C | B | B | NC |
| MG | D | A | B | C | D | F | F | D | C | A | NC |
| JO | D | B | C | C⁻ | D | D⁻ | D⁻ | D⁻ | D⁺ | C | B⁻ |
| TW | C | A⁻ | B⁺ | C⁺ | B⁻ | C | C | B⁺ | A⁻ | A | NC |
| DB | C | A | A | B | C | C | C | B | A | A | NC |

NC - Not completed. Eye comfort recovered the baseline rating prior to initiating game.

The results indicate that all subjects experienced at least a 1 -grade degradation in eye comfort rating within 40 minutes as a result of playing Taipei. Two of the 7 subjects reported a 1 grade degradation in eye comfort; two subjects a 2 grade degradation; two subjects a 3 grade degradation; and one subject a 4 grade degradation. The time required for eye comfort to decline 1 grade varied from 10 minutes to 30 minutes. The magnitude of the degradation varied from 1 to 4 grades.

The creation of high periocular relative humidity when the goggles were worn resulted in an improvement of at least 1 grade for all subjects within 30 minutes.

Three of the 7 subjects reported an improvement of 1 grade of eye comfort within 30 minutes, three subjects a 2-grade improvement, and one subjects an improvement of 4 grades. Only one of the seven subjects required more than 30 minutes to recover to a comfort level equal to their baseline rating prior to initiating computer use with the game Taipei.

It was unexpected that in all cases, a high periocular relative humidity restored eye comfort to the baseline level despite the continuation of the causative adverse circumstances of computer use. All other palliative measures such as blinking, closing the eyes, addition of lubricants, posture changes, etc. at best produce only minimal improvement in comfort if computer use is continued. The majority usually do not achieve an improvement of a complete grade of comfort, as defined in the comfort scale used in these studies, with these conventional measures.

Data for the subjects wearing a swim mask without individual chambers for each eye are presented in following Table III. This data is similar to that found with the swim goggles with individual chambers. TABLE III

TABLE III
EYE COMFORT RATINGS PRIOR TO AND WHEN PLAYING
COMPUTER GAME WITH AND WITHOUT SWIM MASK

| | | | | | | | Ratings Playing Taipei While Wearing | | | | |
|---|---|---|---|---|---|---|---|---|---|---|---|
| | | | | | | | Aqua Sphere Swim Mask To Create | | | | |
| | Usual | | | | | | Higher Levels Of Periocular Relative | | | | |
| | Ratings | Baseline | Ratings Playing Taipei in | | | | Humidity | | | | |
| | During | Ratings | Ambient Room 35-40% | | | | Immmediately | | | | |
| | Routine | Prior to | Relative Humidity | | | | Upon | | | | |
| | Computer | Playing | 10 | 20 | 30 | 40 | Wearing | 10 | 20 | 30 | 40 |
| Subject | Use | Taipei | min | min | min | min | Goggles | min | min | min | min |
| AM | B | B | B | B | C | C | C | B⁺ | B⁺ | A⁻ | A⁻ |
| NB | B | A | B | C | D | D | D | B⁻ | C | B | B⁺ |
| JO | | C− | C− | D+ | D− | | D− | D+ | C+ | B− | |
| EB | | B+ | B+ | B− | C | C− | C− | C | C+ | C+ | B |

The data for subjects wearing the Zeal wrap-around glasses is presented in the following Table IV. This data is better than what would be predicted since the wrap around glasses do not present a sealed chamber and do not create as high a level of periocular humidity as is possible with sealed chambers.

TABLE IV
EYE COMFORT RATINGS PRIOR TO AND WHEN PLAYING
COMPUTER GAME WITH AND WITHOUT WRAP AROUND GLASSES

| | | | | | | Ratings Playing Taipei While Wearing Zeal | | | | | |
|---|---|---|---|---|---|---|---|---|---|---|---|
| | | | | | | Wraparound Glasses Without Individual | | | | | |
| | Usual | | Ratings Playing | | | Chambers For Each Eye To Create Higher | | | | | |
| | Ratings | Baseline | Taipei in Ambient | | | Levels Of Periocular Relative Humidity | | | | | |
| | During | Ratings | Room 35-40% | | | | Immmediately | | | | |
| | Routine | Prior to | Relative Humidity | | | | Upon | | | | |
| | Computer | Playing | 10 | 20 | 30 | 40 | Wearing | 10 | 20 | 30 | 40 |
| Subject | Use | Taipei | min | min | min | min | Goggles | min | min | min | min |
| TD | B⁻ | B⁺ | B⁻ | C | C⁻ | D | D | C⁺ | B | B⁺ | A |
| JO | | B | C+ | D+ | | | D+ | C− | C | C | C |
| EB | | B+ | B | B− | C+ | | C− | C− | C | C+ | B− |

A study was designed to determine whether high relative humidity, approaching 100%, would alter the blink frequency and the innate requirement to blink. The specific steps used for this study follow.

1. The subject and the examiner were seated in a normal work environment with relative humidity of 35 to 40%. The subject was asked to look directly at the eye of the examiner and not to blink. The distance between the subject and the examiner was approximately 3 feet.

2. The subject was requested to report the first burning, tearing, or discomfort of the eyes to the examiner by signaling with the hand. The time when this occurred was then recorded with a stopwatch and recorded as the discomfort interblink time before eyes burned, teared, or suffered discomfort.

3 The time at which the subject was forced to blink was observed and recorded as the “maximum interblink” time. The expected time is 5 to 40 seconds, although in rare instances longer maximum interblink times of 60 to 120 seconds are possible.

4. After a 30-minute rest period, swim goggles as utilized in the studies with the Taipei computer game were fitted, and the latter steps 1 to 3 were repeated. The study was terminated if a blink was not required after 5 minutes.

The results of the above test are set forth in the following Table V.

TABLE V
INTERBLINK TIME IN AMBIENT 40% HUMIDITY AND
IN SWIM GOGGLE CREATING HIGH PERIOCULAR RELATIVE HUMIDITY

| | Normal/Habitual Interblink Times | | | |
|---|---|---|---|---|
| | in Ambient 35-40% Relative | | | |
| | Humidity | | Interblink Times Wearing Swim Goggles | |
| | Discomfort | | Creating High Periocular Humidity | |
| | Interblink | Maximum | Discomfort | |
| | Time (burn, | Interblink Time | Interblink Time | Maximum Interblink Time |
| | tear, | Before Subject | (burn, tear, | Before Subject Forced To |
| Subject | discomfort) | Forced To Blink | discomfort) | Blink |
| TD | 11 sec | 41 sec | 5 mins+ | 5 mins+ |
| DM | 40 sec | 57 sec | 3 min, 5 sec | 4 min, 18 sec |
| JOB | 14 sec | 33 sec | 1 min, 30 sec | 3 min, 16 sec |
| DRK | 12 sec | 15 sec | 4 min, 30 sec | 4 min, 30 sec |
| TW | 5 sec | 30 sec | 5 min+ | 5 min+ |
| NS | 33 sec | 39 sec | 3 min, 45 sec | 5 min+ |
| JME | 19 sec | 34 sec | 5 mins+ | 5 min+ |
| CJK | 07 sec | 09 sec | 3 min, 35 sec | 3 min, 35 sec |
| NB | 24 sec | 31 sec | 5 min+ | 5 min+ |
| VMF | 23 sec | 26 sec | 5 min+ | 5 min+ |

Both the interblink period when burning or discomfort occurred, and the maximum interblink period at which time the subject was forced to blink, were significantly increased for all subjects when wearing the goggles to creates high humidity. This increase for both interblink periods was significant as the usual increase was 5 to 15 times with several subjects achieving over 25 times. Six of the subjects were able to hold their eyes open for 5 minutes, at which time the study was terminated and the goggles removed. The longest maximum interblink time without the goggles was 57 seconds. The median maximum interblink time before the subject was forced to blink was 29 seconds in the normal room environment of 35 -40% humidity. When placed in the swim goggle with high periocular relative humidity, the median forced interblink time increased to over 4 minutes and 30 seconds. Six of the 10 subjects had not been forced to blink for five minutes, at which time the study was terminated.

From the experiments described above, it was concluded that NPIEDS appears to be the result of a combination of, in either order or simultaneously, the excess evaporation from the eye with subsequent sequellae and the thinning of the lipid and other layers of the tear film which occur when a reduced blink frequency is prolonged. The lipid layer can be restored by wearing an appropriate eye-covering device that creates a high level of periocular relative humidity despite a reduced blink rate. With such a device, within 20-40 minutes, eye discomfort induced by near point activity was alleviated. This is as a consequence of the high periocular relative humidity increasing the thickness of the lipid layer thereby compensating for the thinning of the lipid layer induced by the decrease in blinking associated with near point activity. Overcoming NPIEDS by creating high periocular relative humidity when engaged in prolonged near point activity is unexpected as conventional teaching does not suggest that high levels of periocular relative humidity are helpful for eye symptoms induced by such activities. In this respect, the only teaching found in the literature is that moisture chambers can be effective as a part of a dry eye therapy for pathologically dry eyes that require extreme medical treatment. Flynn F. Keratoconjunctivitis sicca and new techniques on its management. Med J Australia 1967; 1 :33; Flynn F. An improved tear-conserving goggle attachment. Med J Australia 1968;2:170; Flynn F. Automatic tear-delivery spectacles—Recent Advances. Australian J Ophthalmol 1975;3:73; 29. Flynn F. A multi-pronged approach in alleviating dry-eye problems. In The Preocular Tear Film in Health, Disease, and Contact Lens Wear. F J Holly, Editor, Dry Eye Institute, Inc. Lubbock, Tex. 1986: 212-220; Rosen J, Brown S I. A simple moist chamber. Am J Ophthalmol 1974; 78:859-860; Poirier R H, Rybum M F, Israel C W. Swimmer's goggles for keratoconjunctivitis sicca. Arch Ophthalmol 1977;95:1405-1406; Savar D A. A new approach to ocular moisture chambers. J Pediatr Ophthalmol Stabismus 1978; 15:51 -53 and Tsubota K, Yamada M, Urayama K. Spectacle side panels and moist inserts for the treatment of dry-eye patients. Cornea 1994; 13: 197-201.

With respect to the design of the device used for practice of the invention—i.e., to create an essentially sealed chamber over the surface of the eye, it requires a physical structure that retains the humidity created by evaporation from the tear film and the surfaces of the skin surrounding the eye so that the periocular relative humidity is increased. The ideal device has individual compartments as present with swim goggles in order to raise the periocular relative humidity in the minimal time and maintain the periocular relative humidity at a high level with means for passage of air and water vapor from each compartment to the outside atmosphere and visa versa to maintain an adequate oxygen supply and to prevent lens fogging as will be explained in greater detail below.

Swim goggles suffer from cosmetic and comfort limitations. In most instances, it is not necessary to add fluid or water vapor to the enclosed chamber since the evaporation from the eye and the surfaces of the skin are adequate to raise the periocular relative humidity to a level adequate to provide comfort. However, in a lesser preferred embodiment of the invention, if desired, water containing inserts can be added to the inner aspect of the eye wear device to supplement the increased humidity and the use of such inserts is within the scope of the invention.

As described above, a relatively high periocular relative humidity is used to increase lipid thickness and to prevent evaporation from the tear film over the eye. In accordance with the invention, a high relative humidity is achieved using a device that encloses the eyes such as any of those devices illustrated in the examples above. The preferred device in accordance with the invention would comprise cosmetically acceptable eyeglasses. Desirably, a flexible appendage or gasket, such as a silicone rubber gasket, would be attached to and surround the rim of the eyeglasses such as to make 360° contact with the face around the area of the eyes to create an almost perfect seal. This appendage could be removable or permanently attached to the frame. The frame should be designed to fit close to the surfaces of the face and minimize the distance of the frame from the surfaces of the face, thereby allowing for a more readily achievable enclosure and moisture barrier. Further, the closer the fitting characteristics of the frame, the more acceptable the appearance.

For those suffering mild to moderate problems, a close fitting frame of a suitable material extending from its inner surface to the face and approximating the facial contours will typically create an adequate increase in the level of periocular relative humidity to provide comfort. For more severe cases, and only as required, water tight chambers may be achieved by attaching flexible silicone rubber to the inner surface of the frame. Several sizes, conforming to the usual spectacle sizes for eye size and nose fit, may be required.

The materials used for the appendage or gasket portions attached to the eyeglasses desirably, in one embodiment of the invention, are permeable to gases as the cornea of the eye is dependent upon oxygen received from the atmosphere. Oxygen permeable materials inclusive of silicone rubber are known in the art. As an alternative to oxygen permeable materials, or for use in combination with such materials, the goggles can be provided with a series of small apertures or openings to facilitate communication of gases within the compartments formed by the goggles and the atmosphere outside of the chambers.

The lenses of the device may be any optical material, coated with appropriate materials to prevent fogging. The lenses may be free of a prescription or may contain the individual patient's lens prescription required for optimal vision as usually practiced. The prescription may also be designed so as to fit on the inner side of the frame as a separate lens system.

When providing a high relative humidity approaching or achieving 100% over the surface of the eye in a confined space, humidity is likely to result in fogging of the inner surface of the lens. The humidity in the goggle is the result of evaporation of fluids from the skin and ocular surfaces. A series of experiments were conducted to determine whether the fogging could be eliminated while maintaining an adequate level of humidity to achieve comfort. These experiments are described below.

Swim goggles of several types were fitted with prescription lenses to correct the vision of the subjects. The first goggles were obtained from Barracuda, Skyline Northwest Corp., Oregon and M0023 goggle, and Franel Optical Supply, Florida. The lenses used were the standard CR-39 ophthalmic plastic lenses. Five subjects wore the swim goggles fitted with these lenses to determine if the inner surface of the lenses would fog, and if so, the time required for the subject to notice clouding or fogginess of vision. When the subject reported fogging of vision, the examiner removed the goggles and inspected the inner surface of the lenses to determine if fogging had occurred. The humidity was measured immediately after the report of fogging by inserting the probe of a Fisherbrand (™) Certified Traceable Digital Hygrometer/Thermometer under the inferior rim of the goggle. Three trials were conducted and the data averaged. In all instances fogging was observed on both lenses. A second goggle was tested using a material developed and provided by General Electric, code name OQ, and reported to be used in ski masks because of anti-fogging capability. The material was obtained in the form of a flat sheet and fabricated into goggles. The experiment described above was repeated with the same subjects and the same type and size of goggle, but with the OQ rather than the CR-39 lenses. The results obtained are set forth in the following table.

TABLE VI
FOG TIME USING OQ LENSE MATERIALS

| | |
|---|---|
| | RESULTS |
| | WITH GENERAL |
| RESULTS WITH CR-39 | ELECTRIC OQ |

| | Time to | Relative | Time to | Relative |
|---|---|---|---|---|
| | first fog | Humidity | first fog | Humidity |
| Subject | (minutes) | (percent) | (minutes) | (percent) |
| DRK | 24 | 85 | 32 | 88 |
| JEK | 14 | 98 | 21 | 100 |
| TD | 12 | 100 | 11 | 100 |

The minimal improvement achieved with the OQ was not adequate to allow the prolonged use of a closed system goggle required for NPIEDS use. Though anti-fog preparations will prevent fogging of lenses for certain applications such as skiing, it has been found that when the level of relative humidity in the enclosed chamber exceeded 80% , the materials were unable to prevent fogging to the extent that they would be suitable for the use contemplated herein.

An additional experiment was conducted to determine the level of relative humidity whereby the inner surface of the OQ lens would fog when worn in a normal room environment. The goggles were modified by drilling circular apertures in to the sides of the plastic housings to allow controlled amounts of humidity to escape, thereby allowing the humidity level to reach a state of equilibrium. Initial studies established that multiple apertures of smaller diameter allowed more precise control than fewer apertures of larger diameter. The following goggles were tested:

Pair 1—4 apertures, each 6 mm in diameter Pair 2—4 apertures, each 4 mm in diameter Pair 3—5 apertures, each 2.5 mm in diameter Pair 4—No apertures, control.

Using the same procedures as those set forth above, the following results were obtained.

TABLE VII
FOG TIME USING GOGGLES PROVIDED WITH APERTURES

| | Pair #1 | | Pair #2 | | Pair #3 | | Pair #4 | |
|---|---|---|---|---|---|---|---|---|
| | (4 apertures, 6 mm) | | (4 apertures, 4 mm) | | (5 apertures, 2.5 mm) | | (no apertures) | |
| | Time | | Time | | Time | | Time | |
| | to 1^{st} | | to 1^{st} | | to 1^{st} | | to 1^{st} | |
| | fog | Humidity | fog | Humidity | fog | Humidity | fog | Humidity |
| Subject | (min) | (%) | (min) | (%) | (min) | (%) | (min) | (%) |
| KL | No | 61/58** | No fog | 91/93 | 30 | 100/100 | 25 | 100/100 |
| | fog* | | | | (OU) | | (OU) | |
| AM | No fog | 66/66 | No fog | 66/65 | 30 | 100/100 | 20 | 90/100 |
| | | | | | (OU) | | (OU) | |
| KO | No fog | 74/77 | No fog | 89/80 | 27 | 100/82 | 10 | 95/96 |
| | | | | | (OD) | | (OU) | |
| JT | No fog | 68/54 | No fog | 86/85 | 30 | 100/92 | 35 | 95/94 |
| | | | | | (OU) | | (OU) | |
| DL | No fog | 85/68 | No fog | 95/87 | 23 | 100/97 | 20 | 100/100 |
| | | | | | (OU) | | (OU) | |
| Averages | | 67.7 | | 83.7 | 22.5 | 97.1 | 22 | 97.0 |

*No fog = no fog with glasses in place for 30 minutes
**Findings for OD/OS (oculus dexter/oculus sinster = right/left)

From the above data, it can be seen that all 5 subject's lenses fogged in the goggle without apertures. The average time to fog was 22.0 minutes, and the relative humidity in the goggle was 97.0%. The goggles with 5 small 2.5 mm. apertures did not change either the time of fogging (22.5 minutes) or the average relative humidity. The goggle with 4 apertures of 4 mm. provided an average humidity of 83.7%, and there was no fogging for any of the 5 subjects over the 90 minutes of the study. The goggle with 4 apertures of 6 mm. diameter provided an average humidity of 67.7% and there was no fogging for any of the 5 subjects over the course of the study. Thus fogging of the OQ lens material could be eliminated at humidity levels in the range of 70 to 85% by appropriate provision of apertures permitting minimal exchange of the atmosphere within the goggles with the outside atmosphere. In addition, the subjects voluntarily reported that they did not have to blink while wearing the goggles, their eyes felt like they had used rewetting eye drops, and that the eyes felt very comfortable for an hour after the goggles were removed.

To confirm that the goggles with the apertures performed in the same manner as those tested above, baseline lipid layer thickness was measured prior to the wearing of the goggles and immediately at the conclusion of the 90 minute study. In all 5 subjects there was a marked increase in lipid layer thickness after wearing each of the 4 pairs of goggles. The data for the pair 4 goggles with no apertures, for pair 2 goggles with 4 apertures of 4 mm diameter, and pair 1 with 4 apertures of 6 mm diameter is provided in table 4.

TABLE VIII
LIPID LAYER THICKNESS USING GOGGLES PROVIDED WITH
APERTURES AT DIFFERING HUMIDITIES

| | Pair 4 | | Pair 2 | | Pair 1 | |
|---|---|---|---|---|---|---|
| | (no apertures) | | (4 apertures, 4 mm) | | (4 apertures, 6 mm) | |
| | (97% Relative | | (83% Relative | | (67% Relative | |
| | Humidity) | | Humidity) | | Humidity) | |
| | Lipid Layer | | Lipid Layer | | Lipid Layer | |
| | Thickness (nm) | | Thickness (nm) | | Thickness (nm) | |
| | | After 90 | | After 90 | | After 90 |
| Subject | Baseline | minutes | Baseline | minutes | Baseline | minutes |
| KL | 60 | 150 | 60 | 180 | 75 | 150 |
| AM | 30 | 90 | 30 | 105 | 90 | 90 |
| KO | 60 | 150 | 90 | 165 | 30 | 90 |
| JT | 30 | 165 | 30 | 150 | 60 | 90 |
| DL | 180 | 180 | 150 | 180 | 105 | 150 |
| Aver- | 72 | 147 | 72 | 156 | 72 | 114 |
| ages | | | | | | |

The maximum thickness possible is 180 nm and the minimum 30 nm. If the baseline lipid layer is 150 to 180, it is not possible to improve the thickness since it is at a maximum to start and cannot be improved. The results of the above experiments establish that there is not a significant difference in lipid layer thickness at the approximate 100% (97%) relative humidity achieved with the pair 4 goggles with no apertures and the 83% relative humidity achieved with pair 2 goggles with 4 apertures of 4 mm. diameter. This finding further establishes that since the increase in lipid layer thickness is the same, the subjective comfort should be the same. However, at 67% relative humidity, the lipid layer thickness, while improved from ambient 35-40% relative humidity, is not optimal.

An experiment was conducted to determine if the OQ lenses in the pair 2 goggles with 4 apertures of 4 mm. in diameter could be worn for the normal work session of 4 hours. Four subjects completed the study. There was no fogging for any of the four subjects during the 4 hours establishing that it is possible to overcome the problem of lens fogging while maintaining adequate levels of humidity.

Six subjects repeated the original study where eye comfort was rated prior to and when playing the computer game Taipei in ambient room conditions of 35-40% relative humidity, and then following the of wearing swim goggles creating high periocular humidity (approaching 100%). The goal of this study was to investigate if there was any difference in eye comfort under these difficult conditions at relative humidity levels varying from approximately 65% to 100%. After 40 minutes of playing the game, the subjects were then immediately fitted on a randomized basis, with either pair 1, 2 or 4 goggles, and the game continued for another 40 minutes. Comfort was rated every 10 minutes. Only one study was conducted on one day. A summary of the results obtained is set forth below.

TABLE IX
COMFORT RATING FOLLOWING GAME PLAY USING
GOGGLES PROVIDED WITH APERTURES

| | Baseline | Comfort | Comfort rating after playing Taipei | | |
|---|---|---|---|---|---|
| | comfort in | rating after | an additional consecutive 40 minutes with | | |
| | 35-40% | playing | goggles | | |
| | relative | Taipei for | | | Pair #1 |
| | humidity | 40 minutes | | | (4 apertures, |
| | prior to | in 35-40% | Pair #4 | Pair #2 | 6 mm) |
| | playing | relative | (no apertures) | (4 apertures, 4 mm) | Approx 67% |
| Subject | Taipei | humidity | Approx. 97% RH | Approx 83% RH | RH |
| 1 | B | C | A | A | C+ |
| 2 | A | D | B | B | C− |
| 3 | A− | C− | A | A− | C− |
| 4 | B | F | B | B | D |
| 5 | A | B | A | A | B |
| 6 | C | F | A | A− | D |

There was no significant difference in the comfort when engaged in the exaggerated use test of the computer game Taipei at the two relative humidities of 83% and 97%. There was an obvious difference at this same activity when the relative humidity was 67%, in that the comfort in all 6 instances was unable to return to the baseline comfort prior to playing the game. With the higher humidity level of 83%, in all instances the comfort level returned to the baseline comfort level, despite the continuation of the adverse circumstance if continuing playing the computer game for an additional 40 minutes. The critical level of periocular relative humidity that allows maximum comfort is over 70%, with these studies supporting a recommendation of over 80%.

With respect to periocular relative humidity within the chamber defined by the goggles and the surface of the face, it should be understood that it will vary from individual to individual. This is due to differing individual rates of evaporation from the skin and ocular surface. Therefore, though generalizations may be made regarding the number of apertures required and their size necessary to maintain periocular relative humidity within the range of 80 to 100 percent without lens fogging, the actual number and their size will vary for each individual. For this reason, it is desirable to provide goggles having means to customize or optimize the transport of water vapor from the chamber defined by the goggles and the surface of face and the outside atmosphere.

Means to customize the transfer of water vapor from the chamber to the atmosphere is readily accomplished using procedures that would be apparent to those skilled in the art. A preferred method would be to provide goggles having a series of preformed sealed apertures prior to use, but readily opened as needed. The preformed apertures are desirably located in the gasket on the sides of the goggles or eyeglasses. The preformed apertures could be opened by perforation such as by piercing the seal covering the aperture with a sharp object such as the point of a ball point pen or punching out the seal with an object such as a ball point pen. The seal over the preformed holes could be a thin plastic film over the surface of the apertures, or perforations around the diameter of a film covering the apertures. The number of such preformed apertures per goggle chamber may vary within wide limits, typically varying in number from about 2 to 10 and preferably from about 3 to 7. The diameter of the preformed apertures may vary within wide limits but preferably does not exceed 10 mm. and more preferably varies between about 2 and 8 mm.

In use of the goggles having the preformed apertures, the user would use the goggles as supplied with the apertures sealed until fogging occurs. Once fogging is encountered, a sealed aperture in each goggle would be perforated. This procedure would be continued until the fogging on the lenses clears.

It should be understood that other methods may be used to create a relatively high humidity over the surface of the eye. For example, a room humidifier could be used to create a relative humidity of 100 percent within the room. However, such an approach is impractical though within the scope of the invention as any means to create the high humidity would alleviate the problems associated with prolonged near point activity.

1. A method for prolonging the period of time during which an individual may engage in near point activity without experiencing visual discomfort, said method comprising the step of maintaining a high relative humidity over the ocular surface of the eyes during at least a major portion of the time during which the individual is engaged in such activity.

2. The method of claim 1 where the high relative humidity is at least 80 percent.

3. The method of claim 1 where the high relative humidity is as close to 100 percent as possible.

4. The method of claim 1 where the near point activity is performed within a distance of from 12 to 36 inches between the eye and the surface read or observed.

5. The method of claim 4 where the distance is between 15 and 24 inches.

6. The method of claim 1 where the near point activity comprises viewing a video display terminal.

7. The method of claim 1 where the near point activity comprises reading.

8. The method of claim 1 where the high relative humidity is maintained by encasing the eyes within a restricted atmosphere.

9. The method of claim 8 where the restricted atmosphere is created by placement of an optically clear barrier over the eyes.

10. The method of claim 9 where the barrier comprises goggles placed over the eyes.

11. The method of claim 9 where the barrier is an eyeglass frame having lenses and a gasket around its rim to create a confined space over the surface of the eyes and means for regulating the relative humidity within the confined space within a range of 80 to 100 percent without fogging of the lenses.

12. The method of claim 12 where the means for regulating the humidity comprises one or more apertures.

13. The method of claim 12 where the apertures are sealed prior to use and during use, the apertures are serially opened until fogging is eliminated.

14. The method of claim 10 where the barrier is coated with an anti fogging agent.

15. A method for prolonging the period of time during which an individual may engage in a visual activity performed within a distance of from 12 to 36 inches between the eye and a surface read or observed without experiencing visual discomfort, said method comprising the step of maintaining a relative humidity of at least 80 percent over the ocular surface of the eyes during at least a major portion of the time during which the individual is engaged in such activity, said relative humidity being maintained by placement of an optically clear barrier over the eyes.

16. The method of claim 15 where the distance between the eye and the surface is between b 15 and 24 inches.

17. The method of claim 15 where the visual activity comprises viewing a video display terminal.

18. The method of claim 15 where the near visual activity comprises reading.

19. The method of claim 15 where the optically clear barrier comprises eyeglasses or goggles having a gasket around its rim to create a confined space over the surface of the eyes, said barrier having lenses coated with an anti fogging agent, said gasket having a sufficient number of apertures within the gasket to permit sufficient transport of water vapor to the atmosphere to prevent fogging while maintaining periocular relative humidity in excess of 80 percent.

20. A device for maintaining a relative humidity of at least 80 percent over the ocular surface, said device comprising lenses and a gasket capable of maintaining a sealed chamber between the surface of the face over the eyes and the atmosphere, said gasket having apertures permitting transport of water vapor between the chamber and the outside atmosphere, said apertures being of sufficient number and size to create a periocular relative humidity of at least 80 percent without fogging of the lenses.

21. The device of claim 20 where the lenses are of an anti-fogging material.

22. The device of claim 20 where the gasket is provided with a series of apertures, some sealed and some open to the atmosphere.

23. The device of claim 22 where the number of apertures does not exceed 10 per chamber.

24. The device of claim 22 where the diameter of the apertures does not exceed 10 mm.

25. The device of claim 24 where the diameter of the apertures varies between 2 and 8 mm.
